(19) United States
(12) Patent Application Publication
(10) Pub. No.: US 2025/0372225 A1
(43) Pub. Date: Dec. 4, 2025

(54) A QUANTITATIVE PHARMACOLOGICAL MODEL OF ENZYME REPLACEMENT THERAPY

(71) Applicant: Denali Therapeutics Inc., South San Francisco, CA (US)

(72) Inventors: Kapil G. Gadkar, Redwood City, CA (US); Mohammad Jafarnejad, Redwood City, CA (US); Robert G. Thorne, San Francisco, CA (US)

(21) Appl. No.: 18/873,764

(22) PCT Filed: Jun. 12, 2023
(86) PCT No.: PCT/US2023/025067

§ 371 Date: Dec. 11, 2024

Related U.S. Application Data
(60) Provisional application No. 63/351,799, filed on Jun. 13, 2022.

(51) Int. Cl. G16H 20/10

(52) U.S. Cl. CPC G16H 20/10

(57) ABSTRACT

This disclosure relates to systems and methods for modeling the pharmacokinetics and pharmacodynamics of enzyme replacement therapies (ERT) in various systems and tissues.

### CLAIM OF PRIORITY

This application claims the benefit of U.S. Provisional Application App. No. 63/351,799, filed on Jun. 13, 2022, the entire contents of which are incorporated herein by reference.

### TECHNICAL FIELD

This disclosure relates to systems and methods for modeling the pharmacokinetics and pharmacodynamics of enzyme replacement therapies (ERT) in various systems and tissues.

### BACKGROUND

Lysosomal storage disorders (LSDs) are relatively rare inherited metabolic diseases that result from defects in lysosomal function. LSDs are typically caused by the deficiency of a single enzyme that participates in the breakdown of metabolic products in the lysosome. The buildup of the product resulting from lack of the enzymatic activity affects various organ systems and can lead to severe symptoms and premature death. The majority of LSDs also have a significant neurological component, which ranges from progressive neurodegeneration and severe cognitive impairment to epileptic, behavioral, and psychiatric disorders. A recombinant form of an enzyme that is deficient in an LSD can be used to treat the disorder (e.g., enzyme replacement therapy, or ERT), but such therapies may have little effect on the brain due to difficulties in delivering the recombinant enzyme across the blood-brain barrier (BBB).

One such example of an LSD is MPS II, also known as Hunter syndrome. Hunter syndrome results from mutations in the gene encoding iduronate-2-sulfatase (IDS), an enzyme responsible for catabolizing glycosaminoglycans (GAGs). IDS deficiency leads to a progressive accumulation of the substrates heparan sulfate and dermatan sulfate throughout the body, with many patients exhibiting cognitive deficits due to accumulation of these toxic substrates in the CNS. The standard of care is a weekly intravenous (IV) infusion of recombinant IDS (Elaprase ®), however, systemic ERT with IDS does not effectively treat the neurocognitive deterioration associated with Hunter syndrome likely due to negligible blood-brain barrier (BBB) permeability and insufficient brain exposure. Monthly intrathecal (IT) IDS administration of recombinant IDS has been tested in the clinic but did not meet its primary endpoint based on a composite cognitive score despite reduction in cerebrospinal fluid (CSF) GAGs.

Ascertaining the pharmacodynamics and pharmacokinetics of ERT treatment and understanding the relationships between clinically measurable biomarkers (e.g., concentrations of substrates and exogenous recombinant enzymes in CSF) and therapeutic efficacy within the affected tissue (e.g., the CNS) can provide valuable insight into future therapeutic development. Therefore, there exists a need for improved methods and systems for modeling the pharmacodynamics and pharmacokinetics of ERT that allow the possibility of predicting the dosing and therapeutic effect of ERT within tissues.

### SUMMARY

This disclosure relates to systems and methods for modeling the pharmacokinetics and pharmacodynamics of enzyme replacement therapies (ERT) in various systems and tissues. In some embodiments, the methods described herein can be used to predict the concentration and/or the effects of therapeutic agents in various tissues, e.g., brain. In some embodiments, the therapeutic agent comprises a TfR-binding moiety.

Provided herein is a method of generating a pharmacokinetic and/or pharmacodynamic profile of a therapeutic agent in a subject, the method comprising using of a model in which a plurality of variables represent a plurality of input rates and output rates for one or more compartments, wherein the therapeutic agent comprises a TfR-binding moiety, wherein the input rate and the output rate of the therapeutic agent depend on the binding affinity of the TfR-binding moiety to TfR and the valency of the TfR-binding moiety.

Also provided herein is a method of determining the efficacy of a therapeutic agent in a subject, with the method including (a) determining a pharmacokinetic and pharmacodynamic profile in the subject, using a model in which a plurality of variables represent a plurality of input rates and output rates for one or more compartments; (b) providing the concentration of the therapeutic agent in a sample collected from the subject; and (c) estimating, based on the concentration of the therapeutic agent in the sample and the pharmacokinetic profile and the pharmacodynamic profile in the subject, the concentration of the therapeutic agent in a tissue. In some embodiments, the one or more compartments include a superficial brain compartment, a deep brain compartment, and a cerebrospinal fluid (CSF) compartment. In some embodiments the superficial brain compartment and the cerebrospinal fluid compartment are connected.

In other embodiments, the one or more compartments also include a CNS endothelial space compartment and a circulation compartment. In some embodiments, the deep brain compartment and the CNS endothelial space compartment are connected. In some embodiments, the CNS endothelial space compartment represents a blood-brain barrier. In some embodiments, the therapeutic agent comprises a TfR-binding moiety.

In some embodiments, the input rate and the output rate of the therapeutic agent depend on the binding affinity of the TfR-binding moiety to TfR and the valency of the TfR-binding moiety. In some embodiments, the tissue is plasma, cerebrospinal fluid, or brain. In some embodiments, the therapeutic agent includes an enzyme. In some embodiments, the enzyme is a lysosomal enzyme. In some embodiments, the enzyme is iduronate-2-sulfatase. In some embodiments, the method includes estimating the concentration of an enzyme substrate and/or an enzyme product in the tissue. In some embodiments, the concentration of the therapeutic agent in the superficial brain compartment is determined by transport of the therapeutic agent from CSF and/or transport of the therapeutic agent across brain endothelium. In some embodiments, the concentration of the therapeutic agent in the superficial brain compartment is calculated by the following equation:

$$\frac{{dD}_{sf}}{dt} = - {k}_{on} \cdot {T}_{sf , bs} \cdot {D}_{sf} + {{k}_{off} [ D T ]}_{sf , bs} + {k}_{ns , brain} \cdot {D}_{C} - {k}_{loss} \cdot {D}_{sf} - {k}_{exchange , D} \cdot ( \frac{{D}_{sf}}{{f}_{ISF 2 B}} - {D}_{CSF} )$$

In some embodiments, the concentration of the therapeutic agent in the deep brain compartment is determined by transport of the therapeutic agent across brain endothelium. In some embodiments, the concentration of the therapeutic agent in the deep brain compartment is calculated by the following equation:

$$\frac{{dD}_{dp}}{dt} = - {k}_{on} \cdot {T}_{dp , bs} \cdot {D}_{dp} + {k}_{off} \cdot {[ DT ]}_{dp , bs} + {k}_{ns , brain} \cdot {D}_{C} - {k}_{loss} \cdot {D}_{dp}$$

In some embodiments, the concentration of the therapeutic agent in the CSF compartment is determined by transport of the therapeutic agent across the blood:CSF barrier and/or exchange of the therapeutic agent with the superficial brain compartment. In some embodiments, the concentration of the therapeutic agent in the CSF compartment is calculated by the following equation:

$$\frac{{dD}_{CSF}}{dt} = {k}_{ns , CSF} \cdot {D}_{C} + {k}_{exchange , D} \cdot ( \frac{{D}_{sf}}{{f}_{ISF 2 B}} - {D}_{CSF} ) - {k}_{loss , CSF} \cdot {D}_{CSF} + {k}_{exchange , SC} \cdot ( {D}_{CSF} - {D}_{SC} )$$

Also provided herein is a method of estimating the concentration of a therapeutic agent in a subject, including (a) determining a pharmacokinetic and pharmacodynamic profile in the subject, using a model in which a plurality of variables represent a plurality of input rates and output rates for one or more compartments, (b) measuring the concentration of the therapeutic agent in a sample collected from the subject, and (c) estimating, based on the concentration of the therapeutic agent in the sample and the pharmacokinetic profile and the pharmacodynamic profile in the subject, the concentration of the therapeutic agent in a tissue.

In some embodiments, the one or more compartments include a superficial brain compartment, a deep brain compartment, a cerebrospinal fluid (CSF) compartment, with the superficial brain compartment and the cerebrospinal fluid compartment being connected. In some embodiments, the one or more compartments include a CNS endothelial space compartment and a circulation compartment, with the deep brain compartment and the CNS endothelial space compartment being connected, and the deep brain compartment and the superficial brain compartment are not connected. In some embodiments, the CNS endothelial space compartment represents a blood-brain barrier. In some embodiments, the therapeutic agent comprises a TfR-binding moiety. In some embodiments, the input rate and the output rate of the therapeutic agent depend on the binding affinity of the TfR-binding moiety to TfR and the valency of the TfR-binding moiety.

Also provided herein is a method of determining the dosage of a therapeutic agent in a subject, including (a) determining a pharmacokinetic and pharmacodynamic profile in the subject, using a model in which a plurality of variables represent a plurality of input rates and output rates for one or more compartments. (b) measuring the concentration of the therapeutic agent in a sample collected from the subject, and (c) estimating, based on the concentration of the therapeutic agent in the sample and the pharmacokinetic profile and the pharmacodynamic profile in the subject, the dosage of the therapeutic agent in the subject.

In some embodiments, the one or more compartments include a superficial brain compartment, a deep brain compartment, a cerebrospinal fluid (CSF) compartment, with the superficial brain compartment and the cerebrospinal fluid compartment being connected. In some embodiments, the one or more compartments include a CNS endothelial space compartment and a circulation compartment, with the deep brain compartment and the CNS endothelial space compartment being connected, and the deep brain compartment and the superficial brain compartment are not connected. In some embodiments, the CNS endothelial space compartment represents a blood-brain barrier. In some embodiments, the therapeutic agent comprises a TfR-binding moiety. In some embodiments, the input rate and the output rate of the therapeutic agent depend on the binding affinity of the TfR-binding moiety to TfR and the valency of the TfR-binding moiety.

Also provided herein is a method of determining the dosage of a therapeutic agent in a subject, including (a) determining a pharmacokinetic and pharmacodynamic profile in the subject, using a model in which a plurality of variables represent a plurality of input rates and output rates for one or more compartments. (b) measuring the concentration of the therapeutic agent in a sample collected from the subject, and (c) estimating, based on the concentration of the therapeutic agent in the sample and the pharmacokinetic profile and the pharmacodynamic profile in the subject, the dosage of the therapeutic agent in the subject.

In some embodiments, the one or more compartments include a superficial brain compartment, a deep brain compartment, a cerebrospinal fluid (CSF) compartment, with the superficial brain compartment and the cerebrospinal fluid compartment being connected. In some embodiments, the one or more compartments include a CNS endothelial space compartment and a circulation compartment, with the deep brain compartment and the CNS endothelial space compartment being connected, and the deep brain compartment and the superficial brain compartment are not connected. In some embodiments, the CNS endothelial space compartment represents a blood-brain barrier. In some embodiments, the therapeutic agent comprises a TfR-binding moiety. In some embodiments, the input rate and the output rate of the therapeutic agent depend on the binding affinity of the TfR-binding moiety to TfR and the valency of the TfR-binding moiety.

Also provided herein is a method of estimating the concentration of an enzyme substrate or an enzyme product in a subject, including (a) determining a pharmacokinetic and pharmacodynamic profile in the subject, using a model in which a plurality of variables represent a plurality of input rates and output rates for one or more compartments. (b) measuring the concentration of the enzyme substrate or enzyme product in a sample collected from the subject, and (c) estimating, based on the concentration of the enzyme substrate or enzyme product in the sample and the pharmacokinetic profile and the pharmacodynamic profile in the subject, the concentration of the enzyme substrate or enzyme product in a tissue.

In some embodiments, the one or more compartments include a superficial brain compartment, a deep brain compartment, a cerebrospinal fluid (CSF) compartment, with the superficial brain compartment and the cerebrospinal fluid compartment being connected. In some embodiments, the one or more compartments include a CNS endothelial space compartment and a circulation compartment, with the deep brain compartment and the CNS endothelial space compartment being connected, and the deep brain compartment and the superficial brain compartment are not connected. In some embodiments, the CNS endothelial space compartment represents a blood-brain barrier. In some embodiments, the therapeutic agent comprises a TfR-binding moiety. In some embodiments, the input rate and the output rate of the therapeutic agent depend on the binding affinity of the TfR-binding moiety to TfR and the valency of the TfR-binding moiety.

Also provided herein is a method of estimating the concentration of an enzyme substrate or an enzyme product in a subject including (a) determining a pharmacokinetic and pharmacodynamic profile in the subject, using a model in which a plurality of variables represent a plurality of input rates and output rates for one or more compartments, (b) measuring the concentration of the enzyme substrate or enzyme product in a sample collected from the subject, and (c) estimating, based on the concentration of the enzyme substrate or enzyme product in the sample and the pharmacokinetic profile and the pharmacodynamic profile in the subject, the concentration of the enzyme substrate or enzyme product in a tissue.

In some embodiments, the one or more compartments include a superficial brain compartment, a deep brain compartment, a cerebrospinal fluid (CSF) compartment, with the superficial brain compartment and the cerebrospinal fluid compartment being connected. In some embodiments, the one or more compartments include a CNS endothelial space compartment and a circulation compartment, with the deep brain compartment and the CNS endothelial space compartment being connected, and the deep brain compartment and the superficial brain compartment are not connected. In some embodiments, the CNS endothelial space compartment represents a blood-brain barrier. In some embodiments, the substrate or enzyme product is a glucosaminoglycan (GAG). In some embodiments, the glucosaminoglycan is heparan sulfate or dermatan sulfate. In some embodiments, the concentration of the substrate or enzyme product depends on the binding affinity of the TfR- binding moiety to TfR and the valency of the TfR-binding moiety.

Also provided herein is a system for performing a method of determining the efficacy of a therapeutic agent in a subject. Also provided herein is a system for performing a method of estimating the concentration of a therapeutic agent in a subject. Also provided herein is a system for performing a method of determining the dosage of a therapeutic agent in a subject. Also provided herein is a system for performing a method of estimating the concentration of an enzyme substrate or an enzyme product in a subject. Also provided herein is a system for performing a method as disclosed herein of generating a pharmacokinetic and/or pharmacodynamic profile of a therapeutic agent in a subject.

Also provided herein is a method for determining response to an enzyme replacement therapy in a subject including (a) modeling a pharmacokinetic profile and a pharmacodynamic profile in the subject using a state variable model in which a plurality of state variables represent a plurality of inputs and outputs of the model, (b) determining the pharmacokinetic profile for the subject based on the state variables of the model, and (c) determining the pharmacodynamic response in the subject based upon the determined pharmacokinetic profile, with the pharmacodynamic response being indicative of response to the enzyme replacement therapy.

In some embodiments, the pharmacodynamic response is a reduction in a glucosaminoglycan (GAG) substrate level relative to a baseline level measured prior to administration of the enzyme replacement therapy. In some embodiments, the pharmacokinetic profile and/or the pharmacodynamic response are determined in the brain. In some embodiments, the pharmacokinetic profile and/or the pharmacodynamic response are determined in the cerebrospinal fluid (CSF). In some embodiments, the model incorporates a parameter based on the ratio of superficial brain:deep brain biodistribution of the enzyme. In some embodiments, the model incorporates a parameter based on the biodistribution of the enzyme in the vascular compartment of the blood-brain barrier. In some embodiments, the model incorporates a parameter based on the biodistribution of the enzyme in the parenchymal compartment of the blood-brain barrier.

In some embodiments, the plurality of state variables include one or more of: (a) route of administration of the ERT, (b) dose amount of the ERT, (c) mannose-6-phosphate receptor (M6PR) binding affinity, and (d) penetration depth of the ERT from CSF. In some embodiments, the plurality of state variables include TfR-binding affinity of the ERT and/or valency of TfR binding of the ERT. In some embodiments, the enzyme target for ERT is a lysosomal storage disease enzyme. In some embodiments, the lysosomal storage disease enzyme is selected from the group consisting of: IDS, SGSH, IDUA, GAA, ARSA, NAGLU, and GCase.

Also provided herein is a method of determining a therapeutically effective dose for an ERT, including (a) modeling a pharmacokinetic profile and a pharmacodynamic profile in the subject using a state variable model in which a plurality of state variables in a state vector represent a plurality of inputs and outputs of the model, (b) determining the pharmacokinetic profile for the subject based on the state variables of the model, and (c) determining the pharmacodynamic response in the subject based upon the determined pharmacokinetic profile, with a change in the pharmacodynamic response in the subject relative to a baseline level being indicative of therapeutic efficacy of the dose.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A a diagram of an enzyme transport vehicle (ETV) for delivery of iduronate-2-sulfatase (IDS) across the blood-brain barrier (BBB), wherein the IDS enzyme is fused to an engineered Fc that includes a transferrin receptor (TfR) binding domain (ETV:IDS).

FIG. 1B is a diagram of bivalent TfR-binding antibody-enzyme fusion with IDS that includes two IDS molecules fused to IgG with TfR-binding Fabs (IgG:IDS).

FIG. 1C is a diagram of intravenous administration of purified recombinant IDS for the treatment of Hunter syndrome (IV:IDS).

FIG. 1D is a diagram of intrathecal administration of purified recombinant IDS for the treatment of Hunter syndrome (IT:IDS).

FIG. 2 is a diagram of predicted penetration depth (mm) from CSF into adjacent brain tissue for mouse (7 mm), macaque (40 mm), and human (120 mm) based on a one-dimension diffusion model.

FIG. 3 is a diagram of the relationships between several compartments involving the central nervous system (CNS) including the systemic circulation, CNS endothelial space, superficial brain tissue, deep brain tissue, and cerebral-spinal fluid (CSF). Arrows indicate connections between compartments and the diffusion of therapeutic agents between compartments.

FIG. 4 is a diagram of the several compartments depicted in FIG. 3, with arrows indicating rates that affect the pharmacokinetics (PK) and pharmacodynamics (PD), of ERT treatment in the CNS.

FIG. 5 is a diagram of the several compartments depicted in FIG. 3, with arrows indicating rates that affect the pharmacokinetics (PK) and pharmacodynamics (PD), of ERT treatment in the CNS with different treatment regimens of IDS as an exemplary ERT.

FIG. 6A is a diagram of the TfR-mediated uptake of delivery of ETV:IDS across the blood-brain barrier (BBB). Rates affecting the enzyme transport vehicle (ETV) delivery of IDS by TfR-mediated transcytosis across the BBB are shown. ETV:IDS includes an IDS molecule fused to an engineered Fc with a monovalent TfR binding domain.

FIG. 6B is an increasingly detailed depiction of the TfR-mediated uptake of delivery of IgG:IDS across the blood-brain barrier (BBB). Rates affecting the IgG delivery of IDS by TfR-mediated transcytosis across the BBB are shown. IgG:IDS includes two IDS molecules fused to IgG with TfR binding Fabs.

FIG. 7A shows results from quantitative system pharmacology (QSP) modeling of four routes of administration of IDS enzyme replacement therapy for Hunter syndrome. Predicted concentration of IDS (nM) is plotted over time for superficial brain (left) and deep brain (center) tissue for clinically relevant doses for the IDS ERT modalities: 3 mg/kg weekly intravenous ETV:IDS; 2 mg/kg weekly intravenous IgG:IDS; 0.5 mg/kg weekly intravenous IDS enzyme; and 10 mg monthly intrathecal IDS enzyme.

FIG. 7B shows results from quantitative system pharmacology (QSP) modeling of four routes of administration of IDS enzyme replacement therapy for Hunter syndrome. Predicted percentage heparan sulfate (HS, an IDS enzyme substrate)) relative to pre-treatment baseline is plotted over time for superficial brain tissue (left), deep brain tissue (center), and total brain (right) for clinically relevant doses for the IDS ERT modalities: 3 mg/kg weekly intravenous ETV:IDS; 2 mg/kg weekly intravenous IgG:IDS; 0.5 mg/kg weekly intravenous IDS enzyme; and 10 mg monthly intrathecal IDS enzyme.

FIG. 7C shows results from quantitative system pharmacology (QSP) modeling of four routes of administration of IDS enzyme replacement therapy for Hunter syndrome. At left, predicted concentration of IDS (nM) in plasma is plotted over time. At right, predicted percentage heparan sulfate (HS, an IDS enzyme substrate) relative to pre-treatment baseline as measured in CSF is plotted over time. Clinically relevant doses for the IDS ERT modalities: 3 mg/kg weekly intravenous ETV:IDS; 2 mg/kg weekly intravenous IgG:IDS; 0.5 mg/kg weekly intravenous IDS enzyme; and 10 mg monthly intrathecal IDS enzyme.

FIG. 8A shows predicted (line) and actual (data points) measurements of plasma PK (left) and total brain PK (right) in mouse for enzyme transport vehicle (ETV) delivery of iduronate-2-sulfatase (IDS). IDS concentration is plotted over time for doses of 1 mg/kg, 3 mg/kg, and 10 mg/kg.

FIG. 8B shows predicted (line) and actual (data points) measurements of plasma PK (left) and total brain PK (right) in mouse for delivery of bivalent TfR-binding IgG-enzyme fusion with IDS. IDS concentration is plotted over time for doses of 1 mg/kg, 3 mg/kg, and 10 mg/kg.

FIG. 8C shows the ratio of parenchymal and vascular PK predicted from the QSP model (line) and measured from preclinical experiments (data points) for ETV:IDS and IgG:IDS in a TfR knock-in mouse at a dose of 10 mg/kg administered intravenously. Ratio of parenchymal:vascular PK is plotted over time.

FIG. 9A shows QSP modeling of equivalent doses of ETV:IDS and IgG:IDS in a human patient at 3 mg/kg dosed weekly. Predicted IDS concentration (nM) in plasma is plotted over time.

FIG. 9B shows QSP modeling of equivalent doses of ETV:IDS and IgG:IDS in a human patient at 3 mg/kg dosed weekly. At left, predicted IDS concentration (nM) in brain vasculature is plotted over time. At center, predicted IDS concentration (nM) in parenchymal brain is plotted over time. At right, predicted parenchymal:vascular ratio of IDS concentration in brain is plotted over time.

FIG. 9C shows QSP modeling of equivalent doses of ETV:IDS and IgG:IDS in a human patient at 3 mg/kg dosed weekly. At left, predicted IDS concentration (nM) in deep brain tissue is plotted over time. At center, predicted IDS concentration (nM) in superficial brain tissue is plotted over time. At right, predicted IDS concentration (nM) in total brain tissue is plotted over time.

FIG. 10A is a bar graph of predicted reductions of heparan sulfate (HS) in cerebral spinal fluid (CSF) for four iduronate-2-sulfatase (IDS) enzyme replacement therapy (ERT) modalities: weekly intravenous dosing of ETV:IDS, IgG:IDS, and purified IDS at 0.5 mg/kg, 1 mg/kg and 2 mg/kg, respectively, and intrathecal dosing of purified IDS at a flat dose of 10 mg (equivalent to 0.34 mg/kg for 30 kg subject). CSF HS can be measured clinically.

FIG. 10B is a bar graph of predicted reductions of heparan sulfate (HS) in brain tissue for four iduronate-2-sulfatase (IDS) enzyme replacement therapy (ERT) modalities: weekly intravenous dosing of ETV:IDS, IgG:IDS, and purified IDS at 0.5 mg/kg, 1 mg/kg and 2 mg/kg, respectively, and intrathecal dosing of purified IDS at a flat dose of 10 mg (equivalent to 0.34 mg/kg for 30 kg subject).

FIG. 11 shows a representative computer system for implementation of the quantitative system pharmacology (QSP) modeling disclosed herein.

### DETAILED DESCRIPTION

Diseases and disorders due to a deficiency or absence of an enzyme in the body can be treated by enzyme replacement therapy (ERT), wherein a medical treatment replaces the enzyme that is deficient or absent, thereby increasing the concentration of the enzyme and alleviating the symptoms of disease. Disorders for which ERT is available and has been shown to be effective include Gaucher disease, Fabry disease, mucopolysaccharidosis (MPS) I, MPS II (Hunter syndrome), MPS VI, and Pompe disease, which are all lysosomal storage diseases. Recombinant enzymes for ERT can be produced in continuous human (fibroblasts) or animal cell lines (Chinese hamster ovary (CHO) cells) and plant cells and can be a purified form of the lysosomal enzymes that is depleted or deficient for the specific disease being treated. Efficacy and safety of ERT for the treatment of multisystem progressive inborn errors of metabolism have been confirmed in clinical trials and clinical practice.

ERT for lysosomal storage disorders (LSD) can include administration of a functional version of the defective enzyme. For example, in Hunter syndrome, iduronate-2-sulfatase (IDS) can be administered to a subject. Following administration, the enzyme can be delivered to the target cells, where it breaks down its substrate in lysosomes, thereby ameliorating the symptoms of the LSD.

### Lysosomal Storage Disorders

Lysosomal storage disorders are inherited metabolic diseases that are characterized by an abnormal build-up of various toxic substance in the body's cells as a result of enzyme deficiencies. There are nearly 50 of these disorders altogether, and they may affect different parts of the body, including the skeleton, brain, skin, heart, and central nervous system. Lysosomal storage disorders include e.g., Sphingolipidoses, Ceramidase, Farber disease, Krabbe disease, Galactosialidosis, Gangliosides: gangliosidoses, Alpha-galactosidase, Fabry disease (alpha-galactosidase A), Schindler disease (alpha-galactosidase B), Beta-galactosidase/GM1 gangliosidosis, GM2 gangliosidosis, AB variant, Activator deficiency, Sandhoff disease, Tay-Sachs, Juvenile hexosaminidase A deficiency, Chronic hexosaminidase A deficiency, Glucocerebroside, Gaucher disease (e.g., Type I, Type II, Type III), Sphingomyelinase, Lysosomal acid lipase deficiency, Niemann-Pick disease (e.g., Type A, Type B), Sulfatidosis, Metachromatic leukodystrophy, Saposin B deficiency, Multiple sulfatase deficiency, Mucopolysaccharidoses, MPS I Hurler syndrome, MPS I S Scheie syndrome, MPS I H-S Hurler-Scheie syndrome, Type II Mucopolysaccharidoses (Hunter syndrome), Type III Mucopolysaccharidoses (Sanfilippo syndrome), Type IV (Morquio), Type VI (Maroteaux-Lamy syndrome), Type VII (Sly syndrome), Type IX (hyaluronidase deficiency), Mucolipidosis (e.g., Type I (sialidosis), Type II (I-cell disease), Type III (pseudo-Hurler polydystrophy/phosphotransferase deficiency), Type IV (mucolipidin 1 deficiency)), Lipidoses, Niemann-Pick disease, Neuronal ceroid lipofuscinoses, Type 1 Santavuori-Haltia disease/infantile NCL (CLN1 PPT1), Type 2 Jansky-Bielschowsky disease/late infantile NCL (CLN2/LINCL TPP1), Type 3 Batten-Spielmeyer-Vogt disease/juvenile NCL (CLN3), Type 4 Kufs disease/adult NCL (CLN4), Type 5 Finnish Variant/late infantile (CLN5), Type 6 Late infantile variant (CLN6), Type 7 CLN7, Type 8 Northern epilepsy (CLN8), Type 8 Turkish late infantile (CLN8), Type 9 German/Serbian late infantile (unknown), Type 10 Congenital cathepsin D deficiency (CTSD), Wolman disease, Oligosaccharide, Alpha-mannosidosis, Beta-mannosidosis, Aspartylglucosaminuria, Fucosidosis, Lysosomal transport diseases, Cystinosis, Pycnodysostosis, Salla disease/sialic acid storage disease, Infantile free sialic acid storage disease, Glycogen storage diseases, Type II Pompe disease, Type IIb Danon disease, Cholesteryl ester storage disease, Lysosomal disease, etc.

Mucopolysaccharidoses (MPS) are a group of metabolic disorders caused by the absence or malfunctioning of lysosomal enzymes needed to break down molecules called glycosaminoglycans (GAGs). These long chains of sugar carbohydrates occur within the cells that help build bone, cartilage, tendons, corneas, skin and connective tissue. GAGs are also found in the fluids that lubricate joints.

Individuals with mucopolysaccharidosis either deficient for one of the eleven enzymes required to break down GAGs, or the enzyme is dysfunctional due to deleterious mutation. Over time, GAGs can collect in cells, blood and connective tissues. The result is permanent, progressive cellular damage which affects appearance, physical abilities, organ and system functioning.

The mucopolysaccharidoses are part of the lysosomal storage disease family. Lysosomes break down this unwanted matter via enzymes, highly specialized proteins essential for survival. Lysosomal disorders like mucopolysaccharidosis are triggered when a particular enzyme exists in too small an amount or is missing altogether.

The various types of MPS have differences and similarities in genetic etiology and clinical presentation, however, the objective for ERT is the same for each disorder: reducing glycosaminoglycan (GAG) accumulation and organomegaly, improving growth (by ameliorating bone structure) and reducing bone deformities, improving the range of motion (ROM) of joints, and improving respiratory function, heart function, hearing, visual acuity, and quality of life for affected patients. The major drawback of ERT molecules is their inability to cross the BBB and cure CNS-associated pathologies and symptoms.

The demonstration that ERT is biochemically effective can be measured by measurement of a target engagement substrate in fluids. For example, in MPS II, levels of GAG can be measured as a biomarker in various sources, including for example, urine, CSF, and plasma.

Mucopolysaccharidosis II (Hunter syndrome) is a rare X-linked recessive lysosomal storage disease caused by deficiency of the lysosomal enzyme iduronate sulfatase (IDS), leading to progressive accumulation of GAGs in nearly all cell types, tissues, and organs. Patients with Hunter syndrome excrete excessive amounts of chondroitin sulfate B (dermatan sulfate) and heparitin sulfate (heparan sulfate) in the urine. Hunter syndrome is a multisystem disorder. Clinical manifestations include severe airway obstruction, skeletal deformities, cardiomyopathy, and, in most patients, neurologic decline. CNS-associated symptoms of the disease can range in severity and include intellectual disability, progressive neurological decline, delayed or absent speech, and seizures Death usually occurs in the second decade of life, although some patients with less severe disease have survived into their fifth or sixth decade.

ERT has been effective for treating the symptoms of Hunter syndrome, wherein recombinant IDS is administered to patients by intravenous or intrathecal injection in order to ameliorate the disease-causing IDS deficiency. Clinical improvements associated with ERT treatment of Hunter syndrome include reduced liver and spleen size, increased forced vital capacity on pulmonary function testing, reduction in the left ventricular mass index, reduction in mortality, and improved quality of life. However, currently available ERT does not treat the cognitive deterioration associated with the disease.

### Blood-Brain Barrier

The use of protein-based therapies to treat diseases in the brain (e.g., certain lysosomal storage disorders in the brain) has been limited by minimal brain exposure following systemic administration. Most polar small molecules and nearly all macromolecules are effectively restricted from reaching the brain in therapeutically relevant concentrations by physical and biochemical barriers, most notably the blood-brain barrier. Brain endothelial cells that form the BBB have several unique physiological properties that distinguish them from peripheral endothelial cells, including tight junctions, relatively low endocytic activity, and the expression of numerous transporters and receptors. As a result. central nervous system (CNS) concentrations of, for example antibodies, often reach only about 0.01-0.1% of peripheral levels after systemic administration, and typically, much of the brain-associated antibody is confined to the endothelium and not parenchymal cells.

The major proportion of the infused recombinant enzymes for treating disease by ERT, for example MPS II (Hunter syndrome), is delivered to the visceral organs such as the liver, kidney, and spleen. The infused enzymes have a short half-life in the circulation due to various factors including degradation, metabolism of the recombinant enzyme, rapid binding of the recombinant enzyme to receptors, and uptake into visceral organs. In most cases only a small fraction of the recombinant enzyme can reach other tissues or organ systems, for example, the bone cartilage and the eye, explaining why improvements of these organ/systems are limited even after long-term treatment. Moreover, due to the inability of recombinant enzymes to cross the blood-brain barrier (BBB), there are typically little to no benefit of ERT for disease symptoms involving the central nervous system (CNS).

The lack of BBB transport of biologic drugs, in general, is a challenge to the treatment of diseases and disorders affecting the CNS, e.g., Hunter syndrome, due to the fact that biologics are large molecule drugs that do not cross the BBB. Attempts can be made to treat the CNS with a variety of BBB avoidance strategies, including intra-thecal (IT) delivery of the enzyme into the cerebrospinal fluid (CSF), stem cell transplant, adeno-associated virus (AAV) gene therapy, or small molecules. Alternatively, BBB drug delivery vehicles can be used. For example, the recombinant enzyme that is deficient in the disease can be re-engineered as an enzyme fusion protein that binds to an endogenous BBB peptide receptor-mediated transport (RMT) system (e.g., insulin receptor or transferrin receptor). RMT is an endogenous process wherein essential biomolecules that cannot passively diffuse into the brain from the bloodstream are actively transported across brain endothelial cells via specific receptors on their luminal surface. The receptor-specific enzyme fusion protein binds an exofacial epitope on the extracellular domain of the endogenous BBB receptor, and this binding can trigger RMT of the fusion protein across the BBB.

Transferrin receptor (TfR) is a carrier protein for transferrin. It is needed for the import of iron into the cell and is regulated in response to intracellular iron concentration. TfR imports iron by internalizing the transferrin-iron complex through receptor-mediated endocytosis. There are two transferrin receptors in humans, transferrin receptor 1 and transferrin receptor 2. Both these receptors are transmembrane glycoproteins. TfR1 is a high affinity ubiquitously expressed receptor while expression of TfR2 is restricted to certain cell types and is unaffected by intracellular iron concentrations. TfR2 binds to transferrin with a 25-30 fold lower affinity than TfR1.

TfR is an effective RMT target at the BBB, owing in part to its enriched expression on brain endothelial cells and its constitutive ligand-independent endocytosis. Multiple platforms targeting TfR have been described, including conventional high-affinity bivalent antibodies, bispecific antibodies, antibody fragments, peptides, antibody-fusion architectures, and an enzyme transport vehicle (ETV) consisting of an Fc domain engineered to directly bind TfR.

Thus, in some embodiments, the therapeutic agent comprises a TfR binding moiety. In some embodiments, the TfR binding moiety is an engineered Fc that includes a TfR-binding domain. In some embodiments, the TfR binding moiety is an antibody or antigen binding fragment thereof that can bind to the TfR, e.g., through antigen binding fragment such as VHH, VH-VL. The use of TfR binding moieties to transport proteins into the brain are described, e.g., in WO 2019/070577, which is incorporated herein by reference in its entirety.

The present disclosure provides methods of predicting PK/PK for a therapeutic agent that cross the blood brain barrier. In some embodiments, the therapeutic agent comprises a TfR binding moiety.

### PK/PD Modeling

For the systems and methods disclosed herein, the pharmacokinetics and pharmacodynamics of a therapeutic agent (e.g., for an enzyme replacement therapy (ERT)) can be predicted on the basis of appropriate variables and rates. Specifically, a pharmacokinetic parameter related to an ERT can be calculated as a predicted value (predicted pharmacokinetic parameter). The calculated predicted pharmacokinetic parameter can be utilized in the determination of the therapeutic effect of the ERT. In some embodiments, an appropriate dose of the ERT to achieve a therapeutically effective reduction in substrate concentration can be determined by determining the therapeutic effect of the ERT on the basis of the predicted pharmacokinetic parameters.

The variables and rates in the context of a tissues (e.g., brain, plasma, CSF etc.) can be regarded as explanatory variables (dependent variables) when the predicted pharmacokinetic parameter is used as an objective variable. In other words, the predicted pharmacokinetic parameter which is an objective variable can be calculated according to a prediction expression with the predetermined background factors and the predetermined gene polymorphisms in the patient as explanatory variables. In this context, the pharmacokinetics of the ERT means disposition related to, particularly, the uptake and elimination of the ERT (e.g., the enzyme or the therapeutic agent comprising the enzyme), the transportation of the ERT, the degradation of the ERT, and/or the association/disassociation with the receptor.

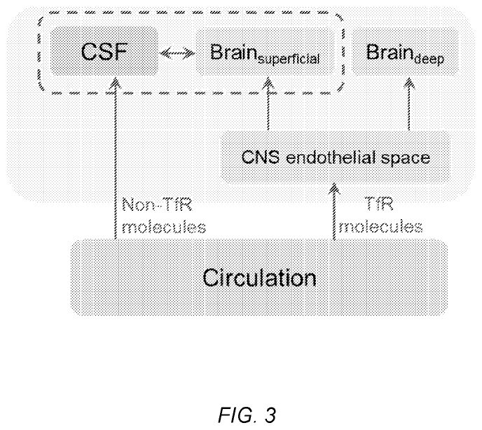

In some embodiments, the PK/PD can be predicted based on the relationship as shown in FIG. 3. As shown in FIG. 3, the circulation compartment is connected with the CNS endothelial space compartment. When a compartment is connected with another compartment, it indicates that the therapeutic agent or some other agents of interest can be transferred between the two compartments. A rate equation can be provided to describe the transfer rate.

In some embodiments, the transfer is bidirectional. In some embodiments, the transfer is unidirectional. The therapeutic agent carrying the transferrin receptor (TfR) binding moiety can cross the blood brain barrier. Thus, in one aspect, the disclosure provides a method of predicting PK/PD for a therapeutic agent with TfR binding moiety or a method of predicting PK/PD for a therapeutic agent in the CNS (e.g., brain). In some embodiments, the CNS endothelial space compartment is then connected with the brain compartment. The brain compartment is connected with the CSF compartment. In some embodiments, two brain compartments are provided: the superficial brain compartment and the deep brain compartment. In some embodiments, only the superficial brain compart is connected with the CSF compartment. In some embodiments, the CSF compartment is connected with the circulation compartment.

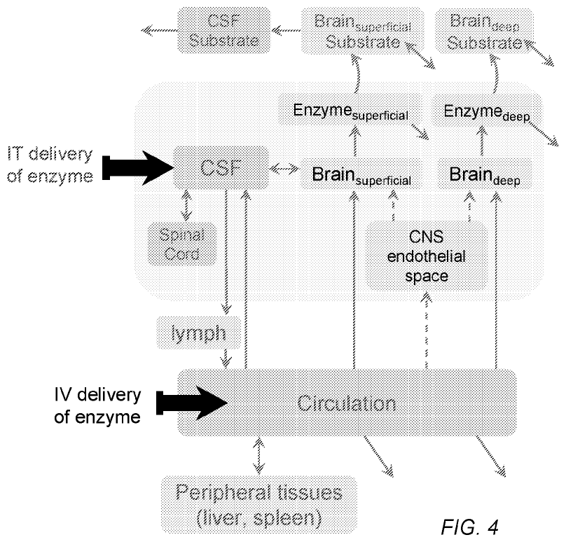

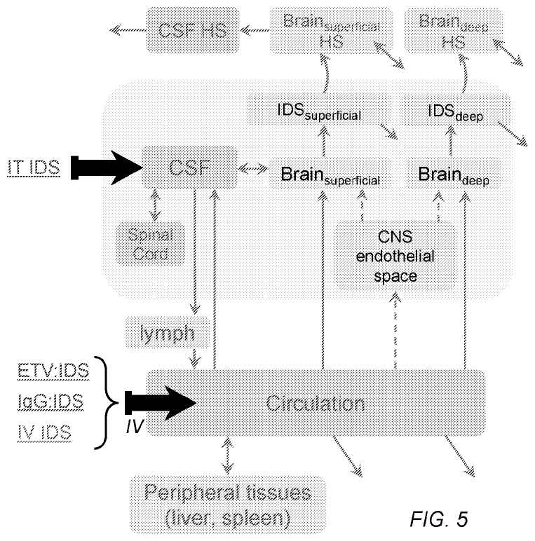

FIG. 4 provides additional information regarding the relationship of different compartments in a more comprehensive model. As shown in FIG. 4, the CSF compartment can be connected with the spinal cord compartment. In addition, the therapeutic agent in the CSF compartment can be cleared by CSF turnover, and transferred to the lymph system compartment, which is further connected with the circulation compartment. In some embodiments, the circulation compartment is connected with peripheral tissues. Furthermore, the therapeutic agent can be cleared by non-specific clearance and/or TfR mediated clearance. In certain embodiments, TfR-mediated clearance shares some parameters with CNS endothelial cell binding, consequently allowing calibration of the model in circulation to inform brain uptake of the therapeutic agent.

The rate of uptake or elimination can be described in equations with various terms. In some embodiments, the rate of concentration change of the therapeutic agent in the superficial brain compartment is determined by the transport of the therapeutic agent from CSF (e.g.,

$${k}_{exchange , D} \cdot ( \frac{{D}_{sf}}{{f}_{ISF 2 B}} - {D}_{CSF} ) )$$

and/or transport of the therapeutic agent across brain endothelium (e.g., k_{ns,brain}·D_{C}). In some embodiments, the rate of concentration change is also determined in part by the association rate of the therapeutic agent with the free receptor on basal BBB (e.g., −kₒₙ·T_{sf,bs}·D_{dp}), and/or the dissociation rate of the drug-receptor complex (e.g., k_{off}·[DT]_{sf,bs}). In some embodiments, the rate is also affected by the uptake rate of the therapeutic agent by tissue cells in the brain (e.g., kₗₒₛₛ·D_{sf}).

In some embodiments, the rate can be represented by the following equation:

$$\frac{{dD}_{sf}}{dt} = - {k}_{on} \cdot {T}_{sf , bs} \cdot {D}_{sf} + {k}_{off} \cdot {[ DT ]}_{sf , bs} + {k}_{ns , brain} \cdot {D}_{C} - {k}_{loss} \cdot {D}_{sf} - {k}_{exchange , D} \cdot ( \frac{{D}_{sf}}{{f}_{ISF 2 B}} - {D}_{CSF} )$$

wherein D represents free intact therapeutic agent (e.g., Enzyme with TfR-binding moiety),
T represents free TfR receptor,
DT represents Drug-TfR complex,
DTT represents TfR-Drug-TfR complex (bivalent bound),
C represents circulation,
P represents peripheral tissues,
CSF represents cerebrospinal fluid,
L represents lymph,
SC represents spinal cord,
sf represents superficial brain,
dp represent deep brain,
ap represents apical BBB,
bs represents basal BBB.

The amount of the enzyme that in the cells in the superficial brain can be determined by the uptake rate and degradation rate. In some embodiments, the rate can be represented by the following equation, wherein E represents enzyme in the therapeutic agent, and n_{E,val} represents valency of the enzyme (number of enzyme units in the therapeutic agent).

$$\frac{{d [ E ]}_{sf}}{dt} = {k}_{loss} \cdot {n}_{E , val} \cdot {D}_{sf} - {k}_{deg , E} \cdot {[ E ]}_{sf}$$

In some embodiments, the rate of concentration change of the therapeutic agent in the deep brain compartment is determined by the transport of the therapeutic agent across brain endothelium (e.g., k_{ns,brain}·D_{C}). In some embodiments, the rate of concentration change is also determined by the association rate of the therapeutic agent with the free receptor on basal BBB (e.g., −kₒₙ·T_{dp,bs}·D_{dp}), and/or the dissociation rate of the drug-receptor complex (e.g., k_{off}·[DT]_{dp,bs}). In some embodiments, the rate is also determined in part by the uptake rate of the therapeutic agent by cells (e.g., kₗₒₛₛ·D_{dp}).

In some embodiments, the rate can be represented by the following equation:

$$\frac{{dD}_{dp}}{dt} = - {k}_{on} \cdot {T}_{dp , bs} \cdot {D}_{dp} + {k}_{off} \cdot {[ DT ]}_{dp , bs} + {k}_{ns , brain} \cdot {D}_{C} - {k}_{loss} \cdot {D}_{dp}$$

The amount of the enzyme that in the cells in the deep brain can be determined by the uptake rate and degradation rate. In some embodiments, the rate can be represented by the following equation, wherein E represents enzyme in the therapeutic agent and n_{E,val} represents valency of the enzyme (number of enzyme units in the therapeutic agent).

$$\frac{{d [ E ]}_{dp}}{dt} = {k}_{loss} \cdot {n}_{E , val} \cdot {D}_{dp} - {k}_{deg , E} \cdot {[ E ]}_{dp}$$

In some embodiments, the rate of concentration change of the therapeutic agent in the CSF compartment is determined by transport of the therapeutic agent across blood:CSF barrier and/or exchange of the therapeutic agent with the superficial brain compartment.

In some embodiments, the rate can be represented by the following equation:

$$\frac{{dD}_{CSF}}{dt} = {k}_{ns , CSF} \cdot {D}_{C} + {k}_{exchange , D} \cdot ( \frac{{D}_{sf}}{{f}_{ISF 2 B}} - {D}_{CSF} ) - {k}_{loss , CSF} \cdot {D}_{CSF} + {k}_{exchange , SC} \cdot ( {D}_{CSF} - {D}_{SC} )$$

From the concentration of the therapeutic agent, the enzyme substrate and/or product can be determined. As shown in FIG. 4, after the therapeutic agent being taken up by cells, the amount of the concentration of the enzyme substrate and/or product can be calculated by the enzyme-catalyzed reaction rate.

In addition, the present disclosure also provides a more accurate model for a therapeutic agent carrying a TfR binding moiety that crosses the blood-brain barrier (BBB). Receptor-mediated transcytosis (RMT) is a vesicular transcellular route by which various macromolecules are transported across a barrier, for example, the blood-brain barrier (BBB). A ligand binding to a receptor on the luminal surface of brain endothelial cells (BEC) triggers ligand-receptor complex endocytosis, routing through various intracellular endosomal compartments where cargo is detached from the receptor and released on the abluminal side, while the receptor recycles ‘back’ to accept additional cargo molecules. The present disclosure provides parameters for a more accurate model of receptor-mediated transcytosis, for example, delivery of enzyme (e.g., IDS) across the BBB for treatment of CNS-associated symptoms and diseases (e.g., Hunter syndrome).

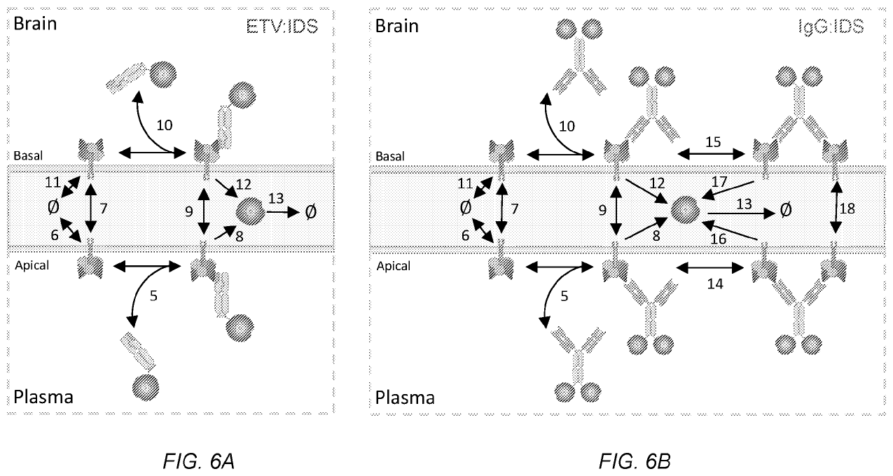

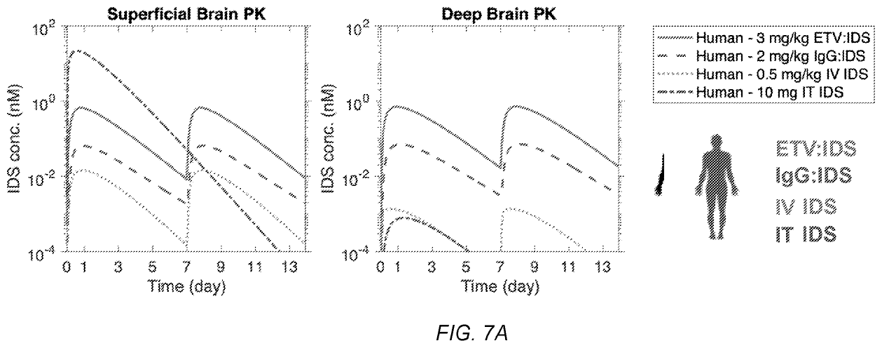

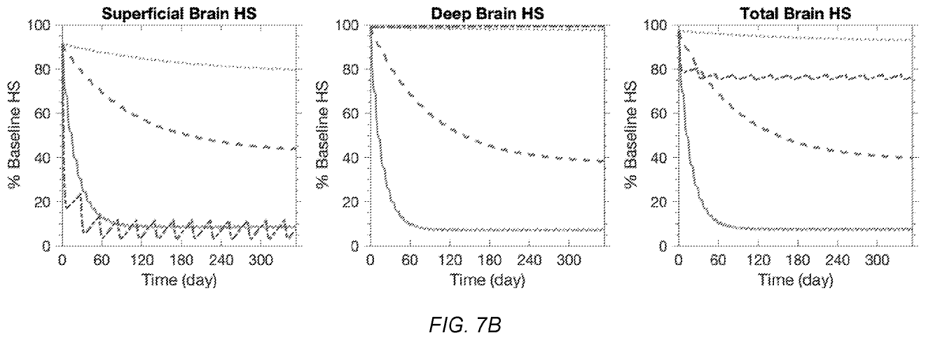

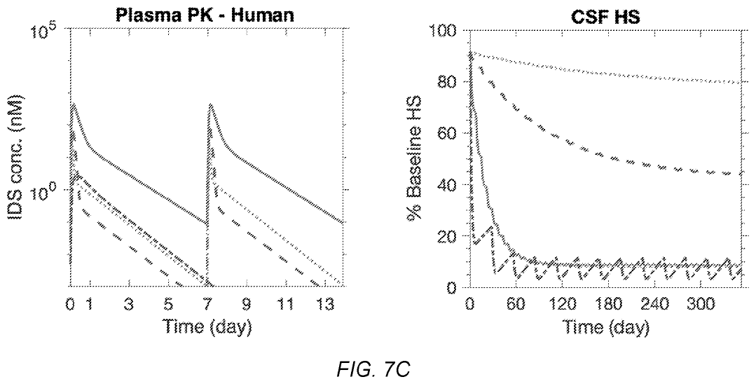

As shown in FIG. 6A, multiple variable rates affect the rate of transport of a therapeutic agent, for example an ETV-delivered enzyme, across the BBB by monovalent binding of the enzyme transport vehicle (ETV) fused to the therapeutic enzyme. FIG. 6A depicts ETV delivery of an enzyme (e.g., IDS) by TfR-mediated transcytosis across the BBB, wherein ETV: Enzyme includes an enzyme (e.g., IDS) molecule fused to an engineered Fc with a monovalent TfR binding domain. Parameters affecting the rate of transcytosis and delivery of the enzyme (e.g., IDS) include affinity of the TfR binding, valency of the antibody-enzyme fusion architecture, antibody dissociation from the receptor upon endocytosis, degradation of the TfR receptor, abundance and concentration of the TfR receptor, concentration of the ETV-enzyme complex, intracellular trafficking of the receptor within the BBB, release of enzyme from BBB endothelial cells into the brain, degradation of the therapeutic enzyme, equilibrium dissociation constant, dissociation rate constant, and association rate constant. Rate variables affecting the transport of IDS across the BBB by TfR-mediated transcytosis include association and dissociation rate between the TfR binding domain and TfR (FIG. 6A, arrow “5” and “10” respectively); rate of ligand-receptor complex transcytosis, endocytosis and intracellular transport (FIG. 6A, arrow “9”); rate of degradation of apical surface ETV:IDS-receptor complex (FIG. 6A, arrow “8”); rate of degradation of basal surface ETV:IDS-receptor complex (FIG. 6A, arrow “12”); rate of receptor transcytosis, endocytosis, recycling, intracellular transport (FIG. 6A, arrow “7”); rate of apical surface receptor degradation (FIG. 6A, arrow “6”); rate of basal surface receptor degradation (FIG. 6A, arrow “11”); rate of degradation of free enzyme within cells of the BBB (FIG. 6A, arrow “13”).

As shown in FIG. 6B, multiple variable rates affect the efficiency of transport of the enzyme (e.g., IDS) across the BBB by multivalent binding (e.g., two binding sites) of an IgG antibody-enzyme fusion protein to one or more TfRs on the apical surface of the BBB. FIG. 6B depicts IgG antibody-mediated delivery of the enzyme (e.g., IDS) by TfR-mediated transcytosis across the BBB, wherein IgG:IDS includes two IDS units fused to an IgG antibody having TfR binding Fabs. Parameters affecting the rate of transcytosis and delivery of the enzyme (e.g., IDS) include affinity of the TfR binding, valency of the antibody-enzyme fusion architecture, antibody dissociation from the receptor upon endocytosis, degradation of the TfR receptor, abundance and concentration of the TfR receptor, concentration of the antibody-enzyme fusion, intracellular trafficking of the receptor within the BBB, release of enzyme from BBB endothelial cells into the brain, degradation of the therapeutic enzyme, equilibrium dissociation constant, dissociation rate constant, and association rate constant. Rate variables affecting the transport of the enzyme (e.g., IDS) across the BBB by TfR-mediated transcytosis of IgG:IDS include association and dissociation constant between the TfR binding domain and TfR (FIG. 6B, arrows “5” and “10” respectively); rate of ligand-receptor complex transcytosis, endocytosis and intracellular transport for a monovalently or bivalently bound complex (FIG. 6B, arrow “9” and arrow “18” respectively); rate of ligand-receptor complex transcytosis, endocytosis and intracellular transport for a bivalently bound complex (FIG. 6B, arrow “18”); association and dissociation constant between the second TfR binding domain of a monovalently bound complex and a second TfR on the apical or basal surface (FIG. 6B, arrow “14” and arrow “15” respectively); rate of degradation of apical surface monovalent or bivalent IgG:Enzyme-receptor complexes (FIG. 6B, arrow “8” and arrow “16” respectively); rate of degradation of basal surface monovalent or bivalent IgG:IDS-receptor complexes (FIG. 6B, arrow “12” and arrow “17” respectively); rate of receptor transcytosis, endocytosis, recycling, intracellular transport (FIG. 6A, arrow “7”); rate of apical surface receptor degradation (FIG. 6B, arrow “6”); rate of basal surface receptor degradation (FIG. 6B, arrow “11”); rate of degradation of free enzyme within cells of the BBB (FIG. 6B, arrow “13”). The model for the transportation of the therapeutic agent at the blood brain barrier can significantly increase the accuracy of the PK/PD prediction for the therapeutic agent in the brain.

In some embodiments, the results from the PD/PK can be used to determine the dosage of the therapeutic agent, including e.g., the dose, the frequency of administration, and the route of administration.

In some embodiments, the methods as described herein further include the methods of treating the subject (e.g., human, non-human animal, mice, monkey) with lysosomal storage disorders.

### Methods of Treatment

In one aspect, the disclosure provides methods for treating a lysosomal storage disorder in a subject, methods of reducing the rate of the lysosomal storage disorder development in a subject over time, methods of reducing the risk of developing a lysosomal storage disorder, or methods of reducing the risk of developing an additional symptom associated with a lysosomal storage disorder in a subject. In some embodiments, the treatment can halt, slow, retard, or inhibit progression of a lysosomal storage disorder. In some embodiments, the treatment can result in the reduction of in the number, severity, and/or duration of one or more symptoms of the lysosomal storage disorder in a subject. In some embodiments, the treatment is based on the PK/PD model or other predictions as described herein.

In one aspect, the disclosure features methods that include administering a therapeutically effective amount of a therapeutic agent thereof disclosed herein (e.g., ERT) to a subject in need thereof (e.g., a subject having, or identified or diagnosed as having, a lysosomal storage disorder).

As used herein, by an “effective amount” is meant an amount or dosage sufficient to effect beneficial or desired results including halting, slowing, retarding, or inhibiting progression of a disease. An effective amount will vary depending upon, e.g., an age and a body weight of a subject to which the agent is to be administered, a severity of symptoms and a route of administration, and thus administration can be determined on an individual basis.

An effective amount can be administered in one or more administrations. By way of example, an effective amount of an agent is an amount sufficient to ameliorate, stop, stabilize, reverse, inhibit, slow and/or delay progression of a disorder in a patient or is an amount sufficient to ameliorate, stop, stabilize, reverse, slow and/or delay toxic material accumulation in a cell in vitro. As is understood in the art, an effective amount of an agent may vary, depending on, inter alia, patient history as well as other factors such as the type (and/or dosage) of the agent used.

Data obtained from cell culture assays and animal studies can be used in formulating an appropriate dosage of any given agent for use in a subject (e.g., a human), e.g., based on the PD/PD model as described herein. A therapeutically effective amount of the therapeutic agent will be an amount that treats the disease in a subject in a subject (e.g., a human subject identified as a lysosomal storage disorder), or a subject identified as being at risk of developing the disease, decreases the severity, frequency, and/or duration of one or more symptoms of a disease in a subject (e.g., a human).

The pharmaceutical compositions can be included in a container, pack, or dispenser together with instructions for administration. The disclosure also provides methods of manufacturing the therapeutic agent for various uses as described herein. In one aspect, the disclosure provides a device that can adjust the dosage of the therapeutic agent based on the PD/PK profile of the therapeutic agent.

### Computer Implementation and Device

The PK/PD model can be stored, e.g., in electronic media such as a flash drive as well as on paper or other media. The PK/PD model can also be represented electronically on a monitor or screen, such as on a computer monitor, a mobile telephone screen, or on a personal digital assistant (PDA) screen. The PK/PD model can also be analyzed and compared by computer in digital, electrical form without the need for a tangible printout or image represented on a computer or other screen or monitor.

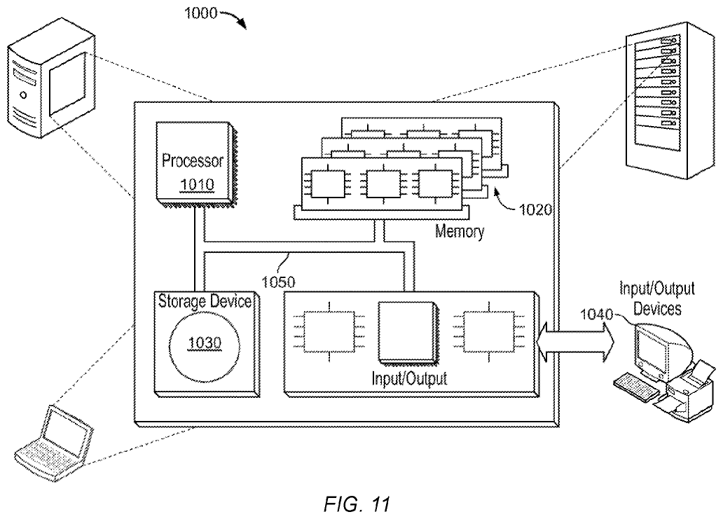

The PK/PD model and the related PK/PD profile can be generated using a computer system, e.g., as described in FIG. 11. FIG. 11 is a schematic diagram of one possible implementation of a computer system 1000 that can be used for the operations described in association with any of the computer-implemented methods described herein. The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. In some embodiments, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In some embodiments, the memory 1020 is a computer-readable medium. The memory 1020 can include volatile memory and/or non-volatile memory.

The storage device 1030 is capable of providing mass storage for the system 1000. In some embodiments, the storage device 1030 is a computer-readable medium. In various different implementations, the storage device 1030 may be a disk device, e.g., a hard disk device or an optical disk device, or a tape device.

The input/output device 1040 provides input/output operations for the system 1000. In some embodiments, the input/output device 1040 includes a keyboard and/or pointing device. In some embodiments, the input/output device 1040 includes a display device for displaying graphical user interfaces.

The methods described can be implemented in digital electronic circuitry, or in computer hardware, software, firmware, or in combinations of them. The methods can be implemented in a computer program product tangibly embodied in an information carrier, e.g., in a machine-readable storage device, for execution by a programmable processor; and features can be performed by a programmable processor executing a program of instructions to perform functions of the described implementations by operating on input data and generating output. The described methods can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Computers include a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, computers and networks that form the Internet.

The computer system can include clients and servers. A client and server are generally remote from each other and typically interact through a network, such as the described one. The relationship of client and server arises by virtue of computer programs running on the respective computers and having a client-server relationship to each other.

The processor 1010 carries out instructions related to a computer program. The processor 1010 may include hardware such as logic gates, adders, multipliers and counters. The processor 1010 may further include a separate arithmetic logic unit (ALU) that performs arithmetic and logical operations.

### EXAMPLES

The invention is further described in the following examples, which do not limit the scope of the invention described in the claims.

### EXAMPLE 1: Quantitative Systems Pharmacology (QSP) Model Predicts Increased Brain Exposure to Iduronate-2-Sulfatase (IDS) by Monovalent Enzyme Transport Vehicle Relative to Other Methods

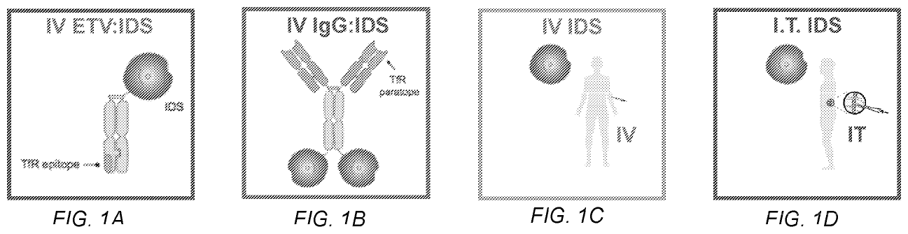

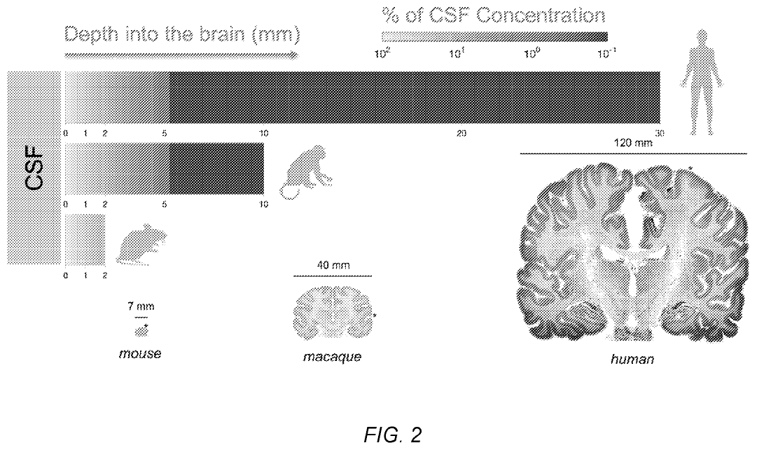

In order to evaluate the quantitative systems pharmacology (QSP) model disclosed herein, four enzyme replacement therapy (ERT) modalities were modeled at doses that are clinically approved or are currently being tested in clinical trials. The four ERT modalities modeled include (1) an intravenously administered enzyme-transport vehicle (IV ETV:IDS), wherein an iduronate-2-sulfatase (IDS) enzyme species is fused to an engineered Fc fragment including a transferrin receptor-binding domain that mediates monovalent binding to the transferrin receptor (TfR) with an affinity of about K_{D}=200 nM (see FIG. 1A); (2) an intravenously administered IgG-mediated delivery of IDS (IV IgG:IDS), wherein two IDS species are fused to an IgG complex comprising TfR binding fragment antigen-binding (Fab) regions that mediate bivalent binding to the TfR with an affinity of about K_{D}=2.6 nM (see FIG. 1B); (3) intravenously administered purified IDS (IV:IDS) (see FIG. 1C); and (4) intrathecally administered purified IDS (IT:IDS) (see FIG. 1D). For the modelling performed in this example, clinically relevant dosing was used as an input for the model. IV ETV:IDS, 3 mg/kg once weekly; IV IgG:IDS, 2 mg/kg once weekly; IV IDS, 0.5 mg/kg once weekly; IT IDS, 10 mg once every four weeks.

QSP modelling results demonstrate superior systemic and brain exposures for IV dosing of IV ETV:IDS compared to both IV IgG:IDS and IV:IDS. IT:IDS results in high exposures of the drug in the cerebrospinal fluid (CSF) and superficial brain but not deep brain. IV ETV:IDS and IV IgG:IDS are taken up by endothelial cells at the blood-brain barrier (BBB) with subsequent TfR-mediated transcytosis facilitating delivery to brain cells, however, the relative efficiency of ETV:IDS transcytosis has been reported to be significantly greater than IgG:IDS transcytosis (Arguello et al. JEM, 2022) resulting in higher enzyme levels in brain with IV ETV:IDS. IV ETV:IDS and IV IgG:IDS architectures access both deep and superficial regions of brain tissue but to varying degrees.

In contrast, IV:IDS enters the brain primarily via the CSF and accesses the superficial brain at low exposures. IT:IDS robustly reduces heparan sulfate (HS) only in the superficial brain region, with negligible HS change in the deep brain region; CSF HS measurements primarily reflect HS lowering in the superficial brain region (and surrounding non-brain meningeal tissues bordering the CSF compartment). IV:IDS is predicted not to produce clinically relevant lowering of HS in brain.

ETV:IDS normalizes HS levels in both superficial and deep brain regions with an HS reduction of ˜90% relative to pre-treatments levels of HS. IgG:IDS results predict HS lowering in brain regions of approximately 50-60% relative to pre-treatments levels of HS due to less efficient delivery of enzyme to parenchymal brain tissue compared to ETV:IDS.

### EXAMPLE 2: QSP Predicts ETV:IDS has Optimal TfR Affinity for Brain Uptake

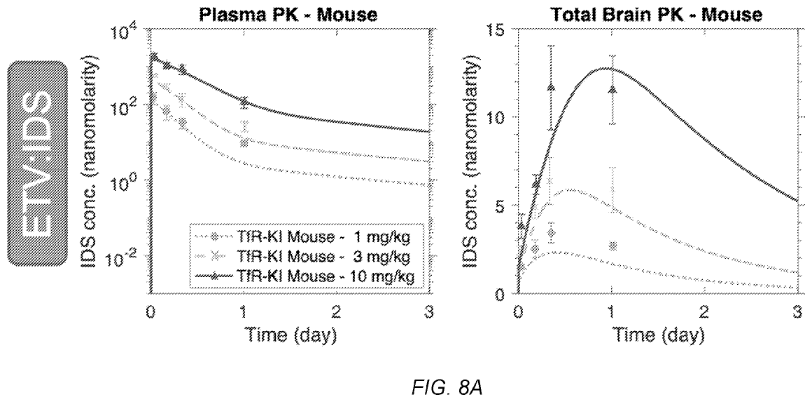

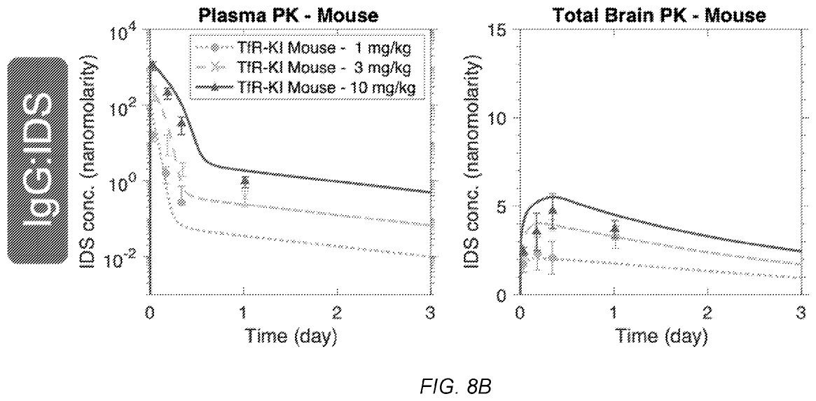

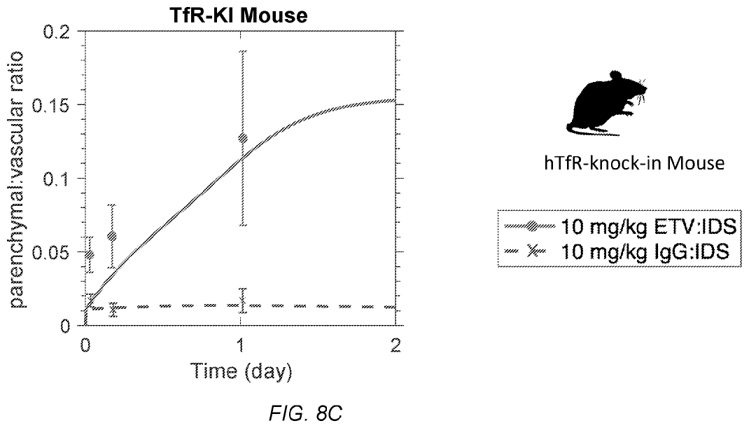

To compare the modelled efficiency of uptake of IDS into brain tissue via ETV:IDS relative to IgG:IDS, systemic and brain pharmacokinetics (PK) were simulated for both ETV and IgG-mediated delivery of IDS at a range of doses. In this example, the model captures preclinical data for systemic and brain PK in a previously published dose ranging study with ETV:IDS and IgG:IDS (Arguello et al. 2022. J Exp Med 219 (3): e20211057). In previous experiments in mouse, as shown in FIG. 8A and FIG. 8B, IgG:IDS demonstrates lower systemic drug PK compared to ETV:IDS due to more pronounced TfR-mediated drug disposition. Brain PK is also improved for ETV:IDS relative to IgG:IDS (FIGS. 8A-8B). The model captures the high affinity, bivalent binding of the IgG:IDS molecule to brain endothelial cell TfR, resulting in entrapment of the molecule in brain endothelial cells and relatively less efficient delivery to the parenchyma compared to ETV:IDS (FIG. 8C). The ETV:IDS molecule monovalently binds TfR with optimized affinity for brain delivery (Ullman et al. 2020. STM 12 (545): eaay1163: Arguello et al. JEM, 2022), resulting in increased distribution to the parenchyma compared to IgG:IDS.

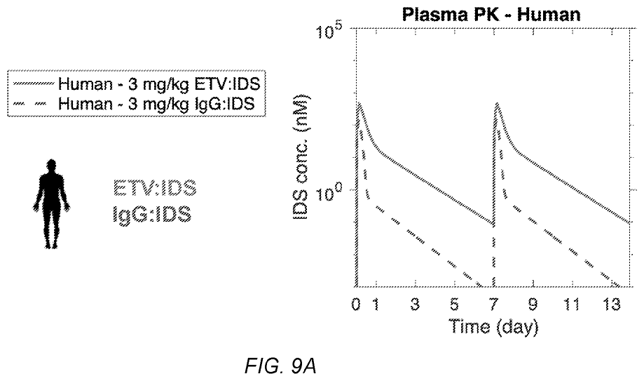

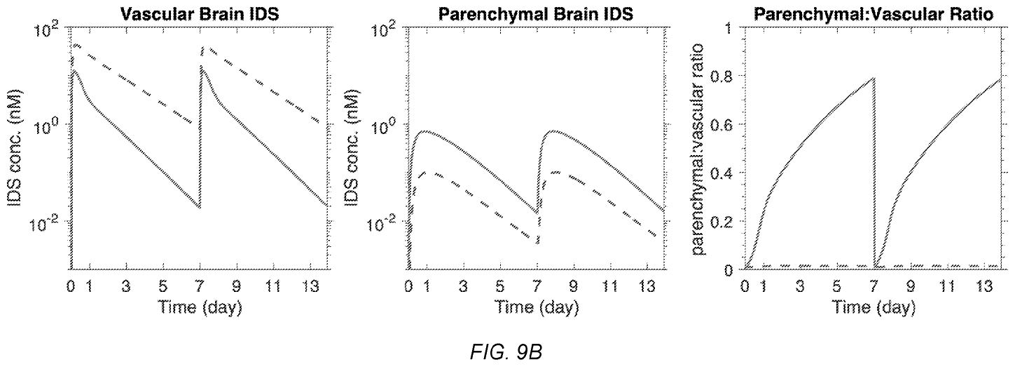

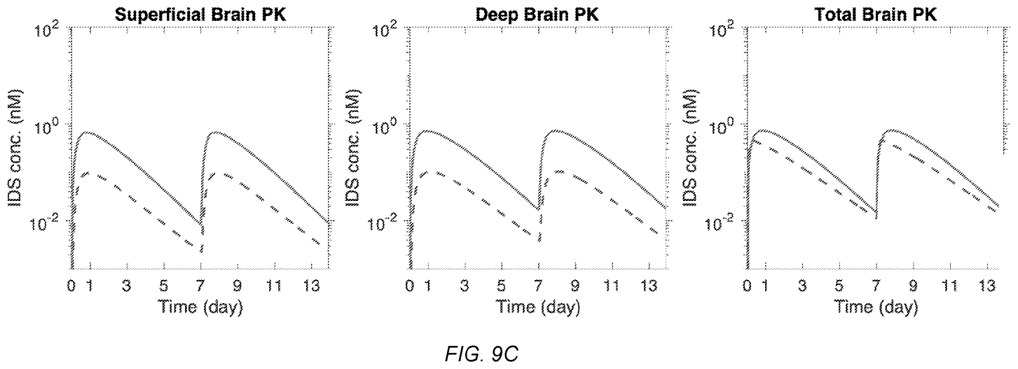

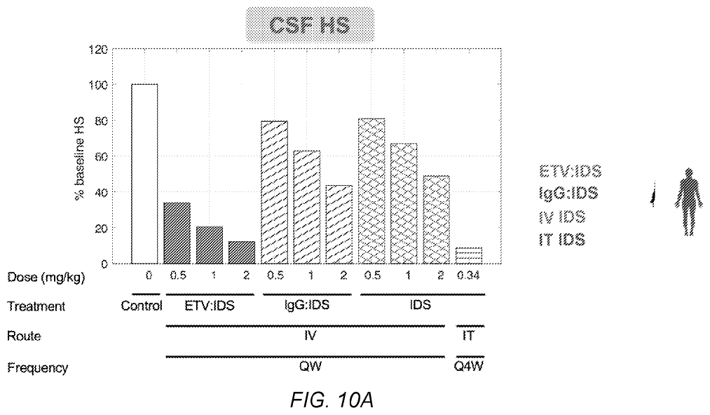

To predict the PK/PD of ETV:IDS and IgG:IDS, with the previous mouse data contributing to the QSP model, human simulations at an arbitrary dose (3 mg/kg QW) predict improved systemic exposure for ETV:IDS relative to IgG:IDS (FIGS. 9A-9C). As shown in FIG. 9B, the simulation predicts IgG:IDS localizes mostly to brain endothelial cells while ETV:IDS distributes to brain parenchyma with much better efficiency. Both ETV:IDS and IgG:IDS utilize TfR-mediated transcytosis and have access to the superficial and deep regions of the brain via the vasculature; however, the optimized TfR interaction of ETV:IDS results in almost 10-fold greater parenchymal brain concentration relative to IgG:IDS, as shown in FIG. 9C.

### EXAMPLE 3: QSP Predicts Effective Reduction of Heparan Sulfate by ETV:IDS in CSF and Brain

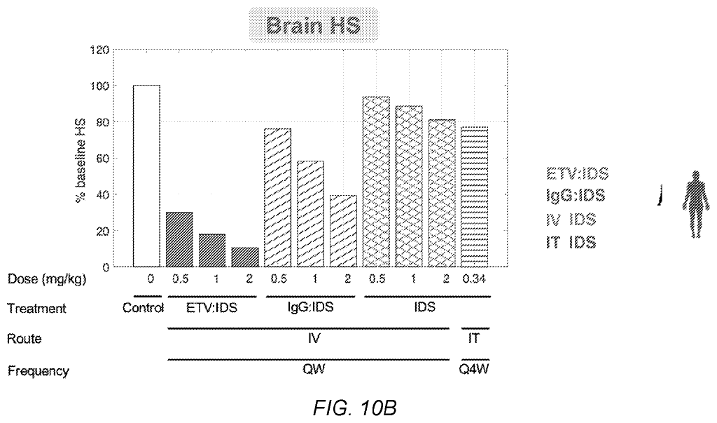

In order to compare the effectiveness of the four modalities for delivery of IDS to brain tissue (ETV:IDS, IgG:IDS, IV:IDS, and IT:IDS) and reduction of heparan sulfate (HS) levels in brain tissue, HS reduction was modelled according to the QSP parameters disclosed herein. The predicted CSF and brain tissue HS reductions with the four different enzyme replacement therapy (ERT) modalities as predicted using the platform QSP models disclosed herein are shown in FIGS. 10A-10B. Human simulations predict that IT:IDS accesses superficial brain well with robust lowering of HS in this region—lowering of HS in superficial brain is reflected in the CSF HS reductions measured; however, there is minimal penetration to deep brain regions and only modest reduction in total brain HS. IV:IDS shows modest dose dependent lowering in CSF HS due to limited activity in superficial brain regions. IDS delivered by IT:IDS and IV:IDS accesses the brain tissue primarily via the CSF (not across the BBB) and therefore penetrates only the superficial brain region; CSF biomarker changes do not reflect the activity of the entire brain tissue due to the lack of penetration into the deep brain region.

In contrast, both ETV:IDS and IgG:IDS access the superficial and deep brain regions to varying degrees via a TfR-mediated brain endothelial cell uptake process and subsequent brain cell delivery dictated by the efficiency of transcytosis events (greater efficiency for ETV:IDS than for IgG:IDS). For these delivery methods, the model simulations disclosed herein can predict the HS reduction in the brain and correlate it with what is measured in the CSF, although actual data indicates that the brain and CSF HS correlations are stronger for ETV:IDS delivery (data not shown). FIG. 10A shows predicted HS reductions in the CSF (clinically measurable) and FIG. 10B shows predicted HS reductions in the brain tissue (clinically unmeasurable) for the IDS ERT modalities: weekly IV dosing of ETV:IDS, IgG:IDS, and IV:IDS at 0.5 mg/kg, 1 mg/kg and 2 mg/kg, and IT:IDS at a flat dose of 10 mg (equivalent to 0.34 mg/kg for 30 kg subject). These simulations are concurrent with preclinical data in the mouse which have suggested that the correlation between CSF and brain GAG reduction is significantly better for ETV:IDS than for IgG:IDS at 7 days following a 1-10 mg/kg single dose (Arguello et al. JEM, 2022).

Taken together, the modeling of PK/PD of the four modalities of IDS ERT (ETV:IDS, IgG:IDS, IV:IDS, and IT:IDS) according to the QSP model disclosed herein indicate that CSF biomarker measurements primarily reflect changes in the superficial brain regions (and CSF bordering non-brain meningeal tissues), and shows that CSF measures are generally not reflective of deep brain activity for non-TfR binding molecules that access brain exclusively or primarily via the CSF. IV:IDS has limited access to brain tissue and is not expected to result in a clinically relevant HS reduction. IT:IDS is anticipated to only access the superficial brain regions in humans and, based on the models, it is not predicted to significantly reduce HS in deep brain regions. TfR-mediated transcytosis facilitates the distribution of ETV:IDS to both the superficial and deep brain regions via the cerebral vasculature. The TfR binding affinity for ETV:IDS has been optimized to maximize the distribution of IDS enzyme to the parenchymal brain region, and robust HS normalization is expected in patients with 3 mg/kg QW dosing of ETV:IDS.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of generating a pharmacokinetic and/or pharmacodynamic profile of a therapeutic agent in a subject, the method comprising using of a model in which a plurality of variables represent a plurality of input rates and output rates for one or more compartments,
wherein the therapeutic agent comprises a TfR-binding moiety, wherein the input rate and the output rate of the therapeutic agent depend on the binding affinity of the TfR-binding moiety to TfR and the valency of the TfR-binding moiety.

2. A method of determining the efficacy of a therapeutic agent in a subject, the method comprising:
a) determining a pharmacokinetic and pharmacodynamic profile in the subject, using a model in which a plurality of variables represent a plurality of input rates and output rates for one or more compartments;
b) providing the concentration of the therapeutic agent in a sample collected from the subject; and
c) estimating, based on the concentration of the therapeutic agent in the sample and the pharmacokinetic profile and the pharmacodynamic profile in the subject, the concentration of the therapeutic agent in a tissue.

3. The method of claim 2, wherein the therapeutic agent comprises a TfR-binding moiety.

4. The method of claim 3, wherein the input rate and the output rate of the therapeutic agent depend on the binding affinity of the TfR-binding moiety to TfR and the valency of the TfR-binding moiety.

5. The method of any one of claims 1-4, wherein the one or more compartments comprise a superficial brain compartment, a deep brain compartment, a cerebrospinal fluid (CSF) compartment, wherein the superficial brain compartment and the cerebrospinal fluid compartment are connected.

6. The method of claim 5, wherein the one or more compartments further comprise a CNS endothelial space compartment and a circulation compartment, wherein the deep brain compartment and the CNS endothelial space compartment are connected.

7. The method of claim 6, wherein the CNS endothelial space compartment represents a blood-brain barrier.

8. The method of any one of claims 2-7, wherein the tissue is plasma, cerebrospinal fluid, or brain.

9. The method of any one of claims 1-8, wherein the therapeutic agent comprises an enzyme.

10. The method of claim 9, wherein the enzyme is a lysosomal enzyme.

11. The method of claim 9, wherein the enzyme is iduronate-2-sulfatase.

12. The method of any one of claims 9-11, wherein the method further comprises estimating the concentration of an enzyme substrate and/or an enzyme product in the tissue.

13. The method of any one of claims 2-12, wherein the concentration of the therapeutic agent in the superficial brain compartment is determined by transport of the therapeutic agent from CSF and/or transport of the therapeutic agent across brain endothelium.

14. The method of claim 13, wherein the concentration of the therapeutic agent in the superficial brain compartment is calculated by
$$\frac{{dD}_{sf}}{dt} = - {k}_{on} \cdot {T}_{sf , bs} \cdot {D}_{sf} + {k}_{off} \cdot {[ DT ]}_{sf , bs} + {k}_{ns , brain} \cdot {D}_{C} - {k}_{loss} \cdot {D}_{sf} - {k}_{exchange , D} \cdot ( \frac{{D}_{sf}}{{f}_{ISF 2 B}} - {D}_{CSF} ) .$$

15. The method of any one of claims 2-14, wherein the concentration of the therapeutic agent in the deep brain compartment is determined by transport of the therapeutic agent across brain endothelium.

16. The method of claim 15, wherein the concentration of the therapeutic agent in the deep brain compartment is calculated by
$$\frac{{dD}_{dp}}{dt} = - {k}_{on} \cdot {T}_{dp , bs} \cdot {D}_{dp} + {k}_{off} \cdot {[ DT ]}_{dp , bs} + {k}_{ns , brain} \cdot {D}_{C} - {k}_{loss} \cdot {D}_{dp} .$$

17. The method of any one of claims 2-16, wherein the concentration of the therapeutic agent in the CSF compartment is determined by transport of the therapeutic agent across blood:CSF barrier and/or exchange of the therapeutic agent with the superficial brain compartment.

18. The method of claim 17, wherein the concentration of the therapeutic agent in the CSF compartment is calculated by
$$\frac{{dD}_{CSF}}{dt} = {k}_{ns , CSF} \cdot {D}_{C} + {k}_{exchange , D} \cdot ( \frac{{D}_{sf}}{{f}_{ISF 2 B}} - {D}_{CSF} ) - {k}_{loss , CSF} \cdot {D}_{CSF} + {k}_{exchange , SC} \cdot ( {D}_{CSF} - {D}_{SC} ) .$$

19. A method of estimating the concentration of a therapeutic agent in a subject, the method comprising:
a) determining a pharmacokinetic and pharmacodynamic profile in the subject, using a model in which a plurality of variables represent a plurality of input rates and output rates for one or more compartments:
b) measuring the concentration of the therapeutic agent in a sample collected from the subject; and
c) estimating, based on the concentration of the therapeutic agent in the sample and the pharmacokinetic profile and the pharmacodynamic profile in the subject, the concentration of the therapeutic agent in a tissue.

20. The method of claim 19, wherein the one or more compartments comprise a superficial brain compartment, a deep brain compartment, a cerebrospinal fluid (CSF) compartment, wherein the superficial brain compartment and the cerebrospinal fluid compartment are connected.

21. The method of claim 19 or 20, wherein the one or more compartments further comprise a CNS endothelial space compartment and a circulation compartment, wherein the deep brain compartment and the CNS endothelial space compartment are connected, and the deep brain compartment and the superficial brain compartment are not connected.

22. The method of claim 21, wherein the CNS endothelial space compartment represents a blood-brain barrier.

23. The method of any one of claims 19-22, wherein the therapeutic agent comprises a TfR- binding moiety.

24. The method of claim 23, wherein the input rate and the output rate of the therapeutic agent depend on the binding affinity of the TfR-binding moiety to TfR and the valency of the TfR-binding moiety.

25. A method of determining the dosage of a therapeutic agent in a subject, the method comprising:
a) determining a pharmacokinetic and pharmacodynamic profile in the subject, using a model in which a plurality of variables represent a plurality of input rates and output rates for one or more compartments;
b) measuring the concentration of the therapeutic agent in a sample collected from the subject; and
c) estimating, based on the concentration of the therapeutic agent in the sample and the pharmacokinetic profile and the pharmacodynamic profile in the subject, the dosage of the therapeutic agent in the subject.

26. The method of claim 25, wherein the one or more compartments comprise a superficial brain compartment, a deep brain compartment, a cerebrospinal fluid (CSF) compartment, wherein the superficial brain compartment and the cerebrospinal fluid compartment are connected.

27. The method of claims 26, wherein the one or more compartments further comprise a CNS endothelial space compartment and a circulation compartment, wherein the deep brain compartment and the CNS endothelial space compartment are connected, and the deep brain compartment and the superficial brain compartment are not connected.

28. The method of claim 27, wherein the CNS endothelial space compartment represents a blood-brain barrier.

29. The method of any one of claims 25-28, wherein the therapeutic agent comprises a TfR- binding moiety.

30. The method of claim 29, wherein the input rate and the output rate of the therapeutic agent depend on the binding affinity of the TfR-binding moiety to TfR and the valency of the TfR-binding moiety.

31. A method of estimating the concentration of an enzyme substrate or an enzyme product in a subject, the method comprising:
a) determining a pharmacokinetic and pharmacodynamic profile in the subject, using a model in which a plurality of variables represent a plurality of input rates and output rates for one or more compartments;
b) measuring the concentration of the enzyme substrate or enzyme product in a sample collected from the subject; and
c) estimating, based on the concentration of the enzyme substrate or enzyme product in the sample and the pharmacokinetic profile and the pharmacodynamic profile in the subject, the concentration of the enzyme substrate or enzyme product in a tissue.

32. The method of claim 31, wherein the one or more compartments comprise a superficial brain compartment, a deep brain compartment, a cerebrospinal fluid (CSF) compartment, wherein the superficial brain compartment and the cerebrospinal fluid compartment are connected.

33. The method of claim 32, wherein the one or more compartments further comprise a CNS endothelial space compartment and a circulation compartment, wherein the deep brain compartment and the CNS endothelial space compartment are connected, and the deep brain compartment and the superficial brain compartment are not connected.

34. The method of claim 33, wherein the CNS endothelial space compartment represents a blood-brain barrier.

35. The method of any one of claims 31-34, wherein the substrate or enzyme product is a glucosaminoglycan (GAG).

36. The method of claim 35, wherein the glucosaminoglycan is heparan sulfate or dermatan sulfate.

37. The method of claim 35 or 36, wherein the concentration of the substrate or enzyme product depends on the binding affinity of the TfR-binding moiety to TfR and the valency of the TfR-binding moiety.

38. A system for performing the method of any one of claims 1-37.

39. A method for determining response to an enzyme replacement therapy in a subject, comprising:
a. modeling a pharmacokinetic profile and a pharmacodynamic profile in the subject using a state variable model in which a plurality of state variables represent a plurality of inputs and outputs of the model;
b. determining the pharmacokinetic profile for the subject based on the state variables of the model; and
c. determining the pharmacodynamic response in the subject based upon the determined pharmacokinetic profile,
wherein the pharmacodynamic response is indicative of response to the enzyme replacement therapy.

40. The method of claim 39, wherein the pharmacodynamic response is a reduction in a glucosaminoglycan (GAG) substrate level relative to a baseline level measured prior to administration of the enzyme replacement therapy.

41. The method of claim 39 or 40, wherein the pharmacokinetic profile and/or the pharmacodynamic response are determined in the brain.

42. The method of any one of claims 39-41, wherein the pharmacokinetic profile and/or the pharmacodynamic response are determined in the cerebrospinal fluid (CSF).

43. The method of any one of claims 39-42, wherein the model incorporates a parameter based on the ratio of superficial brain:deep brain biodistribution of the enzyme.

44. The method of any one of claims 39-43, wherein the model incorporates a parameter based on the biodistribution of the enzyme in the vascular compartment of the blood-brain barrier.

45. The method of any one of claims 39-44, wherein the model incorporates a parameter based on the biodistribution of the enzyme in the parenchymal compartment of the blood-brain barrier.

46. The method of any one of claims 39-45, wherein the plurality of state variables include one or more of:
a. route of administration of the ERT;
b. dose amount of the ERT;
c. mannose-6-phosphate receptor (M6PR) binding affinity; and
d. penetration depth of the ERT from CSF.

47. The method of any one of claims 39-46, wherein the plurality of state variables include TfR-binding affinity of the ERT and/or valency of TfR binding of the ERT.

48. The method of any one of claims 39-47, wherein the enzyme target for ERT is a lysosomal storage disease enzyme.

49. The method of claim 48, wherein the lysosomal storage disease enzyme is selected from the group consisting of: IDS, SGSH, IDUA, GAA, ARSA, NAGLU, and GCase.

50. A method of determining a therapeutically effective dose for an ERT, comprising:
a. modeling a pharmacokinetic profile and a pharmacodynamic profile in the subject using a state variable model in which a plurality of state variables in a state vector represent a plurality of inputs and outputs of the model;
b. determining the pharmacokinetic profile for the subject based on the state variables of the model; and
c. determining the pharmacodynamic response in the subject based upon the determined pharmacokinetic profile;
wherein a change in the pharmacodynamic response in the subject relative to a baseline level is indicative of therapeutic efficacy of the dose.
